(19) United States
(12) Patent Application Publication
(10) Pub. No.: US 2007/0282401 A1
(43) Pub. Date: Dec. 6, 2007

(54) Controller

(75) Inventors: Fred Kahn, Toronto (CA); Eli Hacco, Toronto (CA); Anthony Peter Slonchka, Brampton (CA)

Correspondence Address: GEORGE A. ROLSTON, 45 SHEPPARD AVE EAST, SUITE 900, TORONTO, ON, M2N5W9, US

(73) Assignee: MEDITECH INTERNATIONAL INC

(21) Appl. No.: 11/785,193

(22) Filed: Apr. 16, 2007

Related U.S. Application Data
(60) Provisional application No. 60/795,195, filed on Apr. 27, 2006.

(51) Int. Cl. A61N 5/06

(52) U.S. Cl. 607/88

(57) ABSTRACT

A controller (10) for a light therapy treatment head, providing a portable housing (16, 18), containing a miniaturized control circuit (22) having a CPU (36), and series manually operated controls (20) connected to the CPU, a connection to a power supply (24), and a connection to a light treatment head, whereby the controller may be connected directly to a light treatment head, and may be plugged into a supply of electrical power, and the controller provides control for the operation of the light treatment head, independently of any connection to a computer.

### FIELD OF THE INVENTION

The invention relates to the field of light treatment therapy, and equipment for such therapy.

### BACKGROUND OF THE INVENTION

Light treatment of patients for various conditions is becoming well known. Treatment of injuries such sport injuries brain and the like, treatment of chronic conditions such as arthritis, sciatica and various related conditions, and treatment of chronic slow healing wounds or sores, are all well known.

The principle of all these treatments is the application of low intensity light radiating in the area of the patient's condition. It is found that in order to be effective, the light source should be close in contact with the skin. The light source is usually an array or panel of low intensity light emitting diodes, or in some cases low level laser. It is also found that the treatment becomes more effective over longer treatment periods. The light sources are usually left in contact with the skin for thirty to sixty minutes in many cases. This ensures deep penetration of the light rays into the tissues, and produces the healing results experienced.

In the past, treatment of patients with light therapy has usually involved the connection of the light treatment head to a computer. The computer contained programs for programming the operation of the light treatment head and would regulate the therapy. It would also record the length of therapy and the location and compile records of treatment.

While the system had advantages in the sense of capturing information immediately, it had the disadvantage that the patient was, as it were, tied to the computer. Consequently, the treatment of a single patient would involve a light treatment head, and a dedicated computer. This greatly increased the cost of the light therapy treatment. In addition, it reduced the ability of a clinic to service a number of patients simultaneously. As a result, while the investment in any given clinic, for a given number of patents on a per patient basis was high, the return on investment was low, since it was possible to treat only a limited number of patients at one time.

Clearly, it is desirable to provide a system in which the therapy can be provided by a number of different treatment heads, to a variety of different patients in a given clinic, without the necessity for providing a connection between each treatment head and a computer.

### BRIEF SUMMARY OF THE INVENTION

A controller for a light therapy treatment head, characterised by portable housing containing a miniaturized control circuit having a CPU, and manually operated controls connected to the CPU, a connection to a power supply, and a connection to a light treatment head, whereby the controller may be connected directly to a light treatment head, and may be plugged into a supply of electrical power, so that the controller provides control for the operation of the light treatment head, independently of a computer.

The invention further provides such a controller, having a portable housing which consists in this case, of a lower back panel and upper face panel, which may be mated together to form an enclosure, and in which the enclosure is sized to fit the hand of an operator, and may be slightly in the shape of an hourglass.

The invention further provides such a controller, which further includes a circuit board, on which is mounted a miniature CPU, and other electronic components.
The various features of novelty which characterize the invention are pointed out with more particularity in the claims annexed to and forming a part of this disclosure. For a better understanding of the invention, its operating advantages and specific objects attained by its use, reference should be made to the accompanying drawings and descriptive matter in which there are illustrated and described preferred embodiments of the invention.

### In the Drawings

FIG. 1 is an exploded perspective illustration showing a typical portable controller illustrating the invention, for connection to a light treatment head (not shown), and a connection to a power supply;

FIG. 2 is a schematic bloc diagram illustrating the various components and operations of the controller in controlling a light treatment head.

### DESCRIPTION OF A SPECIFIC EMBODIMENT

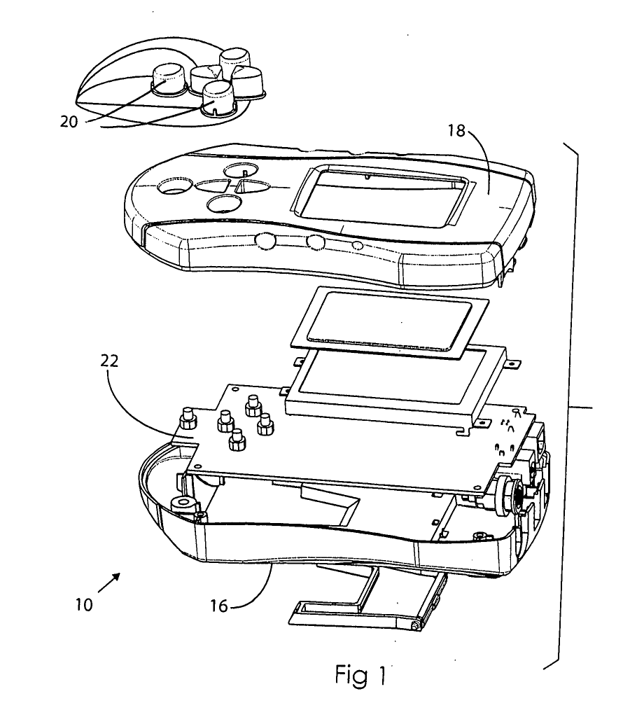

Referring to FIG. 1, it will be seen that the invention is there illustration in the form of a controller (10), which can be connected to a light treatment head not shown, and having a cord (not shown) adapted to be connected to a power supply.

The light treatment head is of the type that incorporates a plurality of light treatment sources, in this case typically light emitting diodes or other forms of lighting source, including laser light, may be provided, and possibly incorporates some form of heat dissipation, whereby heat generated by the lighting sources may be dissipated, and therefore not cause discomfort to the patient.

The controller will be seen to have a simple portable housing which consists in this case, of a lower back panel (16) and upper face panel (18), which may be mated together to form an enclosure. Typically the enclosure will be sized to fit conveniently in the hand of an operator, and may be slightly in the shape of an hourglass, substantially as shown, this being merely one particularly convenient example. Other shapes will suggest themselves to persons skilled in the art for various reasons, and the invention is not restricted to such shape. Within the back panel and face panel, there is a circuit board, on which is mounted a miniature CPU, and various electronic components such as are well known in the art. In addition, there are control buttons (20), in this case three are provided, though whatever number is required would be possible. There could of course be more control buttons. Alternatively there could be a single control button, and mode control, for connecting the single control button to a variety of different mode programs within the device. Such systems are well known in the art and require no special description. The three buttons will typically provide various functions such as an off button, a timer control button, and a power control for adjusting the power supply to the lighting sources in the light treatment head.

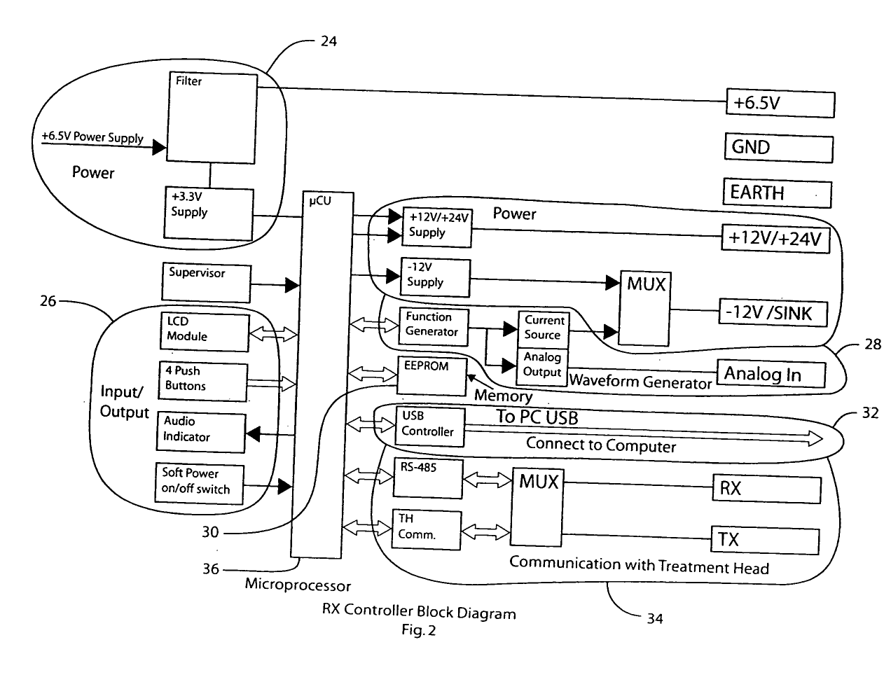

The buttons extend through suitable openings in the face panel (18) of the controller for easy access. The various different control functions are illustrated in FIG. 2, but the invention is not specifically limited to that sequence of control function, various different control functions may be incorporated, without departing from the scope of the invention.

A circuit board (22) housed within panels (16) and (18) provides electronic components for the various operations of the controller.

The main electronic components, and functions of the controller, are as follows. Power supply (24) supplies power for the controller and light treatment head. Input/Output: User interface (26) interfaces the user to control the controller. Waveform Generator (28) creates the waveforms that the treatment head uses. Memory (30) provides local storage for parameters.

Connection (32) allows connection to a computer for firmware and protocol updates. Communication port (34) communicates power with the treatment light head. Microprocessor (36) is the CPU which acts as the brain of the Controller.
The foregoing is a description of a preferred embodiment of the invention which is given here by way of example only. The invention is not to be taken as limited to any of the specific features as described, but comprehends all such variations thereof as come within the scope of the appended claims.

What is claimed is:

1. A manually portable controller (10) for a light therapy treatment head, in which the light treatment head is of the type that incorporates a plurality of light treatment sources, selected from light emitting diodes and laser light sources, and comprising;
a manually portable housing (16, 18);
a miniaturized control circuit (22) within said housing having a CPU;
manually operated controls (20) connected to the CPU;
a connection (24) to a power supply;
a power supply (34) to supply power to a light treatment head, whereby the manually portable controller may be connected directly to a light treatment head, and may be plugged into a supply of electrical power, so that the controller provides both power and control for the operation of the light treatment head.

2. A manually portable controller for a light therapy treatment head as claimed in claim 1, and wherein the housing has a lower back panel (16) and upper face panel, which may be mated together to form said housing.

3. A manually portable controller for a light therapy treatment head as claimed in claim 2 and wherein said housing is sized to fit the hand and is formed in the shape of an hourglass.

4. A manually portable controller for a light therapy treatment head as claimed in claim 3, and which further includes a circuit board(22), within said housing on which is mounted a miniature CPU (36).

5. A manually portable controller for a light therapy treatment head as claimed in claim 4 and which further includes at least one control button (20) for operation of the light treatment head.

6. A manually portable controller for a light therapy treatment head as claimed in claim 5 and including at least one through opening in said face panel of said housing for said at least one button.

7. A manually portable controller for a light therapy treatment head as claimed in claim 1, and wherein said housing contains a miniaturized control circuit (22) having a CPU (36), a memory (30), and a waveform generator (28), and manually operatable controls (20) connected to the CPU, a connection to a power supply (24), and a power supply (34) for connection to a light treatment head, whereby the controller may be connected directly to a light treatment head, and may be plugged into a supply of electrical power, so that the controller provides both power to and control for the operation of the light treatment head.

8. A manually portable controller for a light therapy treatment head as claimed in claim 7, and including two power supplies (24, 34), a first one (24) of said power supplies supplying power to the controller, and a second one (34) of said power supplies supplying power to said light treatment head.

9. A manually portable controller for a light therapy treatment head as claimed in claim 8 and including an input/output user interface (26) which interfaces the user to control the controller, a waveform generator (28) for creating waveforms for use by the light treatment head.

10. A manually portable controller for a light therapy treatment head as claimed in claim 9 and including a memory (30) for local storage for treatment parameters.

11. A manually portable controller for a light therapy treatment head as claimed in claim 10 and further characterised by having a connection (32) for connecting to a computer, whereby to enable connection to the computer for transfer of firmware and protocol updates.
